(19) United States
(12) Patent Application Publication
(10) Pub. No.: US 2012/0283200 A1
(43) Pub. Date: Nov. 8, 2012

(54) PHARMACEUTICAL COMPOSITION AND METHOD FOR PREVENTING OR TREATING CHRONIC HEART DISEASE

(75) Inventors: MING-JAI SU, Taipei (TW); YUEH-HSIUNG KUO, Taipei (TW)

(73) Assignees: CHINA MEDICAL UNIVERSITY, Taichung (TW); National Taiwan University, Taipei (TW)

(21) Appl. No.: 13/431,149

(22) Filed: Mar. 27, 2012

(30) Foreign Application Priority Data
May 3, 2011 (TW) 10011531

(51) Int. Cl. A61K 31/7048; C07C 235/34; C07D 317/58; A61K 31/495; A61K 31/4453; A61P 9/12; A61K 31/36; A61K 31/165; C07H 15/26; A61P 9/00; A61P 9/04; A61P 9/10; C07D 295/192; A61K 31/40

(52) U.S. Cl. 514/25; 544/391; 546/226; 564/170; 549/441; 514/255.01; 514/330; 548/538; 514/423; 514/466; 514/622; 536/18.2

(57) ABSTRACT

The present invention provides a pharmaceutical composition for preventing or treating a chronic heart disease, comprising a compound of a formula (I):

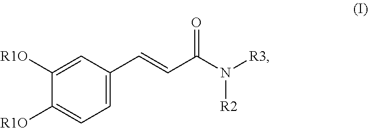

wherein:
R1 is one independently selected from a group consisting of a hydrogen, a methyl and an ethyl;
R2 is one of a hydrogen and a methyl; and
R3 is one selected from a group consisting of a hydrogen, (CH₂)ₙAr and (CH₂)ₙArR′R″, wherein n is one of 1 and 2, R′ and R″ is located at C-3 and C-4 positions, respectively, R′ is a hydrogen and R″ is one of a hydroxy, a fluorine, a bromine and a OMe, or R′+R″=—OCH₂O—; or
R2+R3 is one of

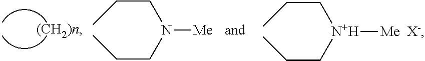

wherein n is one of 4 and 5.

The application claims the benefit of Taiwan Patent Application No. 100115531, filed on May 3, 2011, in the Taiwan Intellectual Property Office, the disclosures of which are incorporated herein in their entirety by reference.

### FIELD OF THE INVENTION

The present invention relates to a catechol-based derivative used for the long-term treatment of chronic heart diseases.

### BACKGROUND OF THE INVENTION

In the past three decades, the citizens in our country were gradually accustomed to western style diet. From then on, the patients having a hypertension and/or metabolic syndromes are increased. These patients have been found to have a higher risk of suffering cardiovascular diseases and a higher mortality rate compared with general population.

A common form of cardiovascular disease is coronary artery disease (CAD), which is resulted from the obstructed or narrowed coronary arteries that supply the myocardium with oxygen and nutrients. CAD is the leading cause of death worldwide, and is associated with the conventional risk factors such as smoking, diabetes, and hypertension.

Coronary heart diseases include heart attacks, sudden unexpected death, chest pain (angina), abnormal heart rhythms, and heart failure due to weakening of the heart muscle. Angina that occurs when an area of the heart muscle doesn't get enough oxygen-rich blood is associated with high grade narrowings of the heart arteries. A heart attack (also known as a myocardial infarction) is the irreversible death of heart muscle from the sudden blockage of a coronary artery by a blood clot. Blockage of a coronary artery deprives the heart muscle of blood and oxygen, causing injury to the heart muscle and may causing the heart failure. Heart failure is generally defined as the inability of the heart to supply sufficient blood flow to meet the needs of the body.

Angiotensin, a peptide, causes blood vessels to constrict, and drives blood pressure up. Angiotensin converting enzyme inhibitors (ACEIs) could cause a vasodilation by inhibiting the formation of angiotensin. Therefore, ACEI critical in the treatment of heart failure are heart medications that widen or dilate the blood vessels to improve the amount of blood the heart pumps, lower the blood pressure and increase the blood flow, which helps to decrease the load of the heart. Further, Angiotensin receptor blockers (ARBs) that block the action of angiotensin by preventing angiotensin from binding to angiotensin receptors on blood vessels could be used to improve the cardiac function in heart failure as well.

Since the above drugs, ACEIs and ARBs, have no direct effect on the abnormal metabolism, a drug effective in treating the Coronary heart diseases caused by the hypertension and the abnormal metabolism is desired.

Hence, because of the defects in the prior arts, the inventors provide a pharmaceutical composition and method for treating chronic heart diseases to effectively overcome the demerits existing in the prior arts.

### SUMMARY OF THE INVENTION

Since a drug effective in treating the coronary heart diseases caused by the hypertension and the abnormal metabolism is desired, the present invention provides a pharmaceutical composition and method for treating chronic heart diseases.

In accordance with one aspect of the present invention, a pharmaceutical composition for preventing or treating a chronic heart disease, particularly a chronic heart failure, is provided. The pharmaceutical composition comprises a compound of a formula (I):

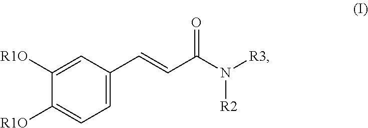

wherein:
R1 is one independently selected from a group consisting of a hydrogen, a methyl and an ethyl;
R2 is one of a hydrogen and a methyl; and
R3 is one selected from a group consisting of a hydrogen, (CH₂)ₙAr and (CH₂)ₙArR′R″, wherein n is one of 1 and 2, R′ and R″ is located at C-3 and C-4 positions, respectively, R′ is a hydrogen and R″ is one of a hydroxy, a fluorine, a bromine and a OMe, or R′+R″=—OCH₂O—; or
R2+R3 is one of

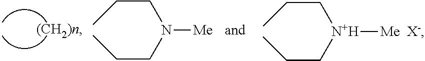

wherein n is one of 4 and 5.

In accordance with another aspect of the present invention, a method of treating or preventing a chronic heart disease is provided. The method comprises a step of administering a therapeutically effective amount of the above mentioned pharmaceutical composition to a subject in need thereof.

In accordance with a further aspect of the present invention, a compound of a formula (II) is provided:

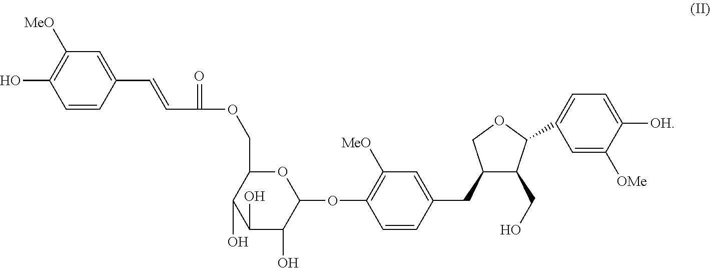

In accordance with a further aspect of the present invention, a pharmaceutical composition for preventing or treating a chronic heart disease is provided. The pharmaceutical composition comprises a compound of the abovementioned formula (II).

The invention provides methods of treatment and prophylaxis by administering to a subject of an effective amount of a composition of the invention. In a preferred aspect, the composition is substantially purified. The subject is preferably an animal, including but not limited to animals such as cows, pigs, horses, chickens, cats, dogs, etc., and is preferably a mammal, and most preferably human. In a specific embodiment, a non-human mammal is the subject.

The present invention also provides pharmaceutical compositions. Such compositions comprise a therapeutically effective amount of a compound, and a pharmaceutically acceptable carrier. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Such compositions will contain a therapeutically effective amount of the compound, preferably in purified form, together with a suitable amount of carrier so as to provide the form for proper administration to the patient. The compounds of the invention can be formulated as neutral or salt forms. The compounds of the invention can be synthetic compounds. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, etc.

The amount of the composition of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. The effective therapeutic dose used of the compounds according to the present invention in animals is comparable or lower than the current clinical drugs, such as ACEI, ARB and carvedilol. There are no obvious side effects found in chronic oral administration. Since the compounds according to the present invention could improve the abnormal metabolism, such as a high blood sugar, without causing a low blood pressure, they could not only used in cardiac dysfunction in heart failure, but also in diabetic cardiovascular complications.

The above objects and advantages of the present invention will become more readily apparent to those ordinarily skilled in the art after reviewing the following detailed descriptions and accompanying drawings, in which:

### BRIEF DESCRIPTION OF THE DRAWINGS

FIGS. 1(a) and 1(b) are diagrams showing the effects of the compounds in the present invention and metformin on the viability of cells treated by the hypoxia-reoxygenation.

FIGS. 2(a) and 2(b) are diagrams showing the effects of the compound 370G provided in the present invention on the heart function parameters, ejection fraction (FIG. 2(a)) and PRSW (FIG. 2(b)).

FIG. 3 is a diagram showing the effects of insulin and the compound 370G on the infarct/ischemic areas of the cardiac muscle of the type I diabetic rats after the ligation/reperfusion treatment.

FIG. 4(a) is a diagram showing the effects of the compound 370G on the infarct/ischemic areas of the cardiac muscle of the type II diabetic mice after the ligation/reperfusion treatment.

FIG. 4(b) is a diagram showing the effects of the compound 370G on the coronary flow rate of the type II diabetic mice after the ligation/reperfusion treatment.

FIG. 5(a) is a diagram showing the effects of the compound 370G on the body weight (BW) of mice treated with the AAB operation.

FIG. 5(b) is a diagram showing the effects of the compound 370G on the heart weight (HW) of mice treated with the AAB operation.

FIG. 5(c) is a diagram showing the effects of the compound 370G on the HW/BW of mice treated with the AAB operation.

FIG. 5(d) is a diagram showing the effects of the compound 370G on the left ventricle weight (LVW) of mice treated with the AAB operation.

FIG. 5(e) is a diagram showing the effects of the compound 370G on the LVW/HW of mice treated with the AAB operation.

FIG. 5(f) is a diagram showing the effects of the compound 370G on the LVW/BW of mice treated with the AAB operation.

FIG. 6(a) is a diagram showing the evaluated results of the cardiac function, dp/dt max, in five groups.

FIG. 6(b) is a diagram showing the evaluated results of the cardiac function, −dp/dt min, in five groups.

FIG. 7(a) is a diagram showing the effect of the compound according to the present invention on the changes in plasma levels of angiotensin II after the aortic constriction.

FIG. 7(b) is a diagram showing the effect of the compound according to the present invention on the changes in plasma levels of ANP after the aortic constriction.

FIG. 8 is a diagram showing the effect of the compound according to the present invention on the changes in the cardiomyocyte diameter after the aortic constriction.

FIG. 9 is a diagram showing the effect of the compound according to the present invention on the changes in the plasma LDH level after the aortic constriction.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

Structural Formulae and Manufacturing Method of Compounds 642C, 642D and 370G

The formulae and the manufacturing method of compounds 642C, 642D and 370G have been disclosed in Taiwan Patent application with the Publication No. 200812566, the contents of which are hereby incorporated by reference herein.

Structural Formulae and Extraction Method of Compounds RS-47 and RS-59

The dry roots (8 kg) of Rhus javanica var. roxburghiane are extracted with MeOH (50 L, each one week two times). The extract is concentrated, the residue (300 g) is suspended in H₂O (2.5 L) and partitioned with AcOEt (3 L×3), and then the aqueous phase is successively partitioned with BuOH (3 L×3). The BuOH extract is concentrated to leave a residue (70 g), which is subjected to a dry column chromatography with silica gel (1.0 kg), wherein CHCl₃/MeOH/H₂O is used as eluent. It gives 13 fractions. Each fraction is subjected to Sephadex LH-20 and RP-18 columns, eluted with CH₃OH/H₂O (10-90%), and finally purified by a silica gel column to obtain RS-59 (3,7,4′-trihydroxyflavone), RS-26 (7,3′,4′-trihydroxyflavone), RS-47 (lariciresinol 4′-(6″-O-feruloyl-β-D-glucopyranoside)), RS-25 (5,7,3′,4′-tetrahydroxyflavone), and RS-28 (3,5,7,3′,4′-pentahydroxyflavone). The data of the physical properties the of the above purified compounds analyzed by the 1H-NMR spectra and high resolution fast atom bombardment mass spectrometry (HR-FAB-MS) are shown in Table 1.

TABLE 1

| | |
|---|---|
| Compound | RS-59 |
| Structural formula | 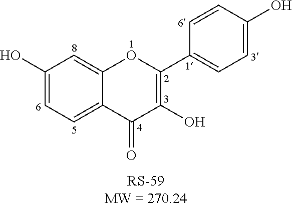 |
| ¹H-NMR | 6.92 (1H, d, J = 2.3 Hz, H-8), 7.92 (1H, d, J = 8.6 Hz, H-5), 6.90 (1H, dd, J = 8.6, 2.3 Hz, |
| (CD₃OD, | H-6), 8.05 (2H, d, J = 9.0 Hz, H-2′, 6′), 6.92 (2H, d, J = 9.0 Hz, H-3′, 5′) |
| 400 MHz) | |
| HR-FAB-MS | 271.2451 [(M + 1)⁺, C₁₅H₁₁O₅⁺] |
| Compound | RS-26 |
| Structural formula | 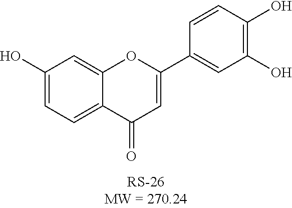 |
| ¹H-NMR | 6.63 (1H, s, H-3), 7.59 (1H, d, J = 8.3 Hz, H-5), 6.72 (1H, dd, J = 8.3, 2.0 Hz, H-6), 6.77 |
| (CD₃OD, | (1H, d, J = 2.0 Hz, H-8), 7.51 (1H, d, J = 2.0 Hz, H-2′), 6.90 (1H, d, J = 8.0 Hz, H-5′), and |
| 400 MHz) | 7.32 (1H, dd, J = 8.0, 2.0 Hz, H-6′) |
| HR-FAB-MS | 271.2456 [(M + 1)⁺, C₁₅H₁₁O₅⁺] |
| Compound | RS-47 |
| Structural formula | 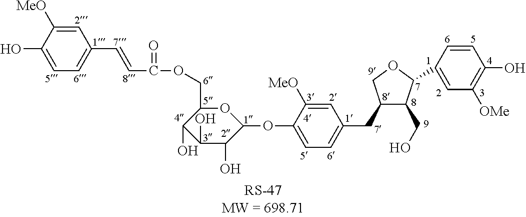 |
| ¹H-NMR | 6.79 (1H, d, J = 2.4 Hz, H-2), 7.12 (1H, d, J = 8.0 Hz, H-5), 6.76 (1H, dd, J = 8.0, 2.4 Hz, |
| (CD₃COOD, | H-6), 4.75 (1H, d, J = 6.0 Hz, H-7), 2.22-2.29 (1H, m, H-8), 3.64, 3.81 (each 1H, |
| 400 MHz) | overlapping with other signals, Hₐ-9, H_{b}-9), 3.79 (3H, s, 3-OCH₃). 6.96 (1H, d, J = 2.0 Hz, |
| | H-2′), 6.72 (1H, d, J = 8.0 Hz, H-5′), 6.32 (1H, dd, J = 8.0, 2.0 Hz, H-6′), 2.88 (1H, dd, |
| | J = 13.4, 4.5 Hz, Hₐ-7′), 2.46 (1H, dd, J = 13.4, 4.8 Hz, H_{b}-7′), 2.61 (1H, m, H-8′), 3.85, |
| | 3.81 (each 1H, overlapping with other signals, Hₐ-9′, H_{b}-9′), 3.79 (3H, s, 3′-OCH₃), 4.90 |
| | (1H, d, J = 7.2 Hz, H-1″), 3.53, 3.59, 3.48, 3.73 (each 1H, H-2″, -3″, -4″, -5″), 4.51 (1H, |
| | dd, J = 12.4, 2.0 Hz, Hₐ-6″), 4.35 (1H, dd, J = 12.4, 6.8 Hz, H_{b}-6″), 7.33 (1H, d, J = 2.4 Hz, |
| | H-2′′′), 6.87 (1H, d, J = 7.6 Hz, H-5′′′), 7.13 (1H, dd, J = 7.6, 2.4 Hz, H-6′′′), 7.61, 6.22 |
| | (each 1H, d, J = 16.0, H-7′′′, H-8′′′), and 3.90 (3H, s, 3′′′-OCH₃) |
| HR-FAB-MS | 699.2659 [(M + 1)⁺, C₃₆H₄₃O₁₄⁺] |
| Compound | RS-25 |
| Structural formula | 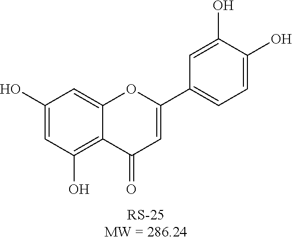 |
| ¹H-NMR | 6.51 (1H, s, H-3), 6.19, 6.42 (each 1H, d, J = 2.0 Hz, H-6, H-8), 7.49 (1H, d, J = 2.2 Hz, |
| (CD₃COOD, | H-2′), 7.00 (1H, d, J = 8.0 Hz, H-5′), 7.45 (1H, dd, J = 8.0, 2.2 Hz, H-6′) |
| 400 MHz) | |
| HR-FAB-MS | 287.2445 [(M + 1)⁺, C₁₅H₁₁O₆⁺] |
| Compound | RS-28 |
| Structural formula | 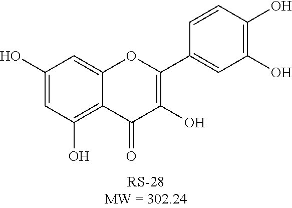 |
| ¹H-NMR | 6.16, 6.35 (each 1H, d, J = 2.0 Hz), 7.65 (1H, d, J = 2.0 Hz, H-2′), 6.68 (1H, d, J = 8.0 Hz, |
| (CD₃COOD, | H-5′), 7.62 (1H, dd, J = 8.0, 2.0 Hz, H-6′) |
| 400 MHz) | |
| HR-FAB-MS | 303.2450 [(M + 1)⁺, C₁₅H₁₁O₇⁺] |

Cell Viability Assay of H9c2 Cell in Hypoxia-Reoxygenation Model

1. Cell Culture

Rat embryonic ventricular myoblast cell line, H9c2, are cultured in a DMEM medium supplemented with 10% fetal bovine serum (FBS), penicillin (10 U/mL), streptomycin (100 μg/mL) and amphotericin B (0.25 μg/mL) in 37° C. humidified 5% carbon dioxide (CO₂) incubator. When the plate becomes confluent, maintain the culture at a 1:4 subculture split.

2. Hypoxia-Reoxygenation Model

(1) H9c2 cells are cultured on a 60 mm plate (6×10⁵ cells/plate) containing 10% FBS DMEM (high glucose DMEM) for 24 hours. The medium is replaced with DMEM with no serum for the starvation. At the same time, a medium containing DMEM with no glucose and no serum is adjusted to pH 6.8 and placed into a hypoxia chamber (95% N₂ and 5% CO₂, 37° C.) where the oxygen partial pressure is controlled to be less than 2% for a pretreatment for 24 hours. (2) After the 24 hours starvation, the culture medium is replaced with the pretreated DMEM with no glucose and no serum, and the plates are placed in the hypoxia chamber to induce hypoxia for 6 hours. (3) After 6 hours, the culture medium is replaced with 10% FBS DMEM and the plates are placed into a growth chamber (5% CO2, 37° C.) for 12 hours, which is so called reoxygenation treatment.

Cells are divided into control, vehicle (sham) and drug groups. In drug group, RS-47 (1 μM, 3 μM, 10 μM), RS-59 (1 μM, 3 μM, 10 μM), 642C (1 μM, 3 μM, 10 μM), 642D (1 μM, 3 μM, 10 μM) and metformin (10 μM, 30 μM, 100 μM) are respectively added into the starvation medium (DMEM with no serum) 1 hour before the hypoxia treatment. For the vehicle group, the hypoxia-reoxygenation treatment is performed without any addition of drugs. For the control group, only the starvation is performed without any other treatments. MTT assay is used to show cell viability of each group.

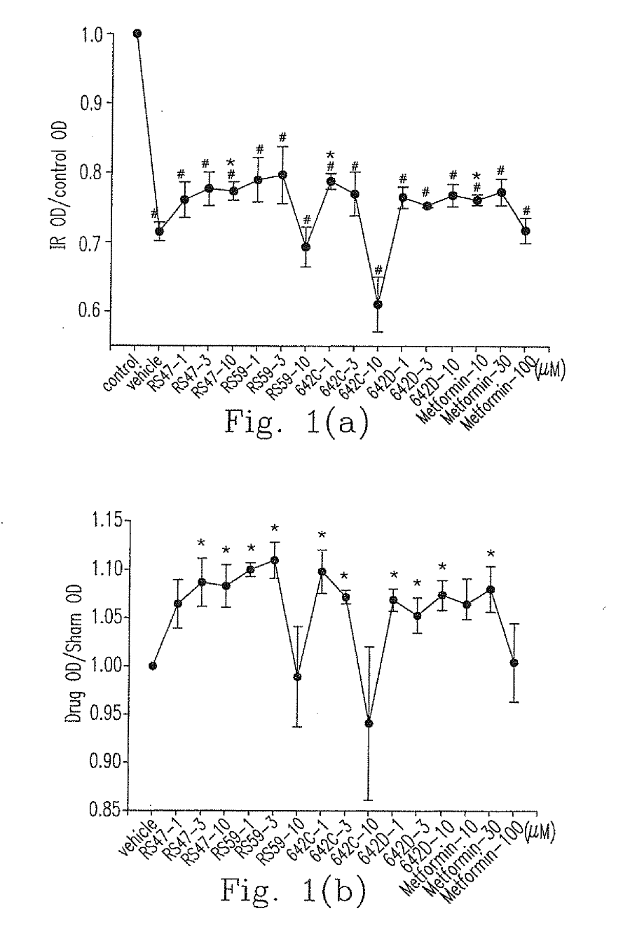

Please refer to FIGS. 1(a) and 1(b), which are diagrams showing the effects of the compounds in the present invention and metformin on the viability of cells treated by the hypoxia-reoxygenation. The results are represented by the absorbance (OD) values of the hypoxia-reoxygenation treated group (IR OD)/the OD values of the control group (control OD) in FIG. 1(a) and the OD values of the drug-treated group (drug OD)/the OD values of the sham group (sham OD). As shown in FIG. 1(b), the viability of the cells treated with the hypoxia-reoxygenation is significantly increased in the drug groups RS-47, 642D and the anti-diabetic medicines, metformin, when compared to the sham group. Therefore, the compounds RS47 and 642D of the present invention significantly increase the cell viability in H9c2 cells suffering from the hypoxia reoxygenation injury, and this is attributed to a decrease of cell apoptosis and necrosis.

Induction of Diabetes in Rats/Mice

1. Induction of Type I Diabetes in Rats Using Streptozotocin (STZ)

After fasting 72 hours, male Wistar rats (8-week old) are anaesthetized by an intraperitoneal injection of a pentobarbital (30 mg/kg, i.p.). Then, they are treated with STZ (60 mg/kg, Sigma Chemical Company, St. Louis, U.S.A.) via an injection into the femoral vein. After four weeks, a blood sample is taken from the veins, and the blood glucose value is obtained via a glucose kit and an analyser (Biosystem S.A., Barcelona Spain, BST330). Rats are considered as the type I diabetic rats when they have the blood glucose exceeding 350 mg/dL and meanwhile have the three symptoms of excessive thirst, excessive urination and excessive appetite.

2. Induction of Type II Diabetes in Mice

Male ICR mice (4-week old) are fed with high-oil diet and fructose solution for 4 weeks. After fasting overnight, blood is drawn through the retro-orbital plexus, and the blood glucose value is obtained via the glucose kit and the analyser. Mice are considered as the type II diabetic mice when they have the blood glucose at least exceeding 150 mg/dL.

Evaluation of the Function of the Left Ventricle

Rats are anesthetized with pentobarbital (30 mg/kg body weight, i.p.). A pressure/volume measuring catheter connected to the signal converter is inserted via the cervical artery into the left ventricle. The converted signals are amplified by an amplifier, and then acquired by a SciSense system so as to display/record the real-time data on/in a computer. The computer is used to calculate the heart function parameters, such as the ejection fraction, preload recruitable stroke work (PRSW), +dp/dt, and −dp/dt.

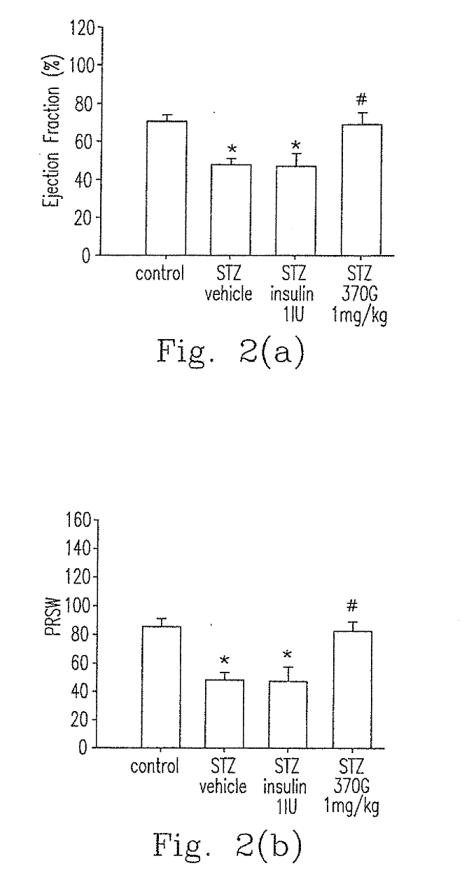

Please refer to FIGS. 2(a) and 2(b), which are diagrams showing the effects of the compound 370G provided in the present invention on the heart function parameters, ejection fraction (FIG. 2(a)) and PRSW (FIG. 2(b)). In this embodiment, the STZ-induced type I diabetic rats are treated with insulin (1 IU/kg, STZ-insulin group), vehicle (STZ-vehicle group) or compound 370G (1 mg/kg, STZ-3700 group). As shown in FIGS. 2(a) and 2(b), the heart function parameters, ejection fraction and PRSW, are significantly increased by the treatment of the compound 370G (* denotes p<0.05 compared with the control group, and # denotes p<0.05 compared with the STZ-vehicle group). That is, the long-term treatment of the compound 370G would effectively improve the function of the left ventricle of the type I diabetic rats.

Evaluation of the Protection Against the Ischemia-Reperfusion Induced Myocardial Injury

1. The methods of the ligation and the reperfusion of the coronary artery of the living rats have been disclosed in Taiwan Patent application with the Publication No. 200812566.

2. The method for detecting the myocardial infarction area has been disclosed in Taiwan Patent application with the Publication No. 200812566.

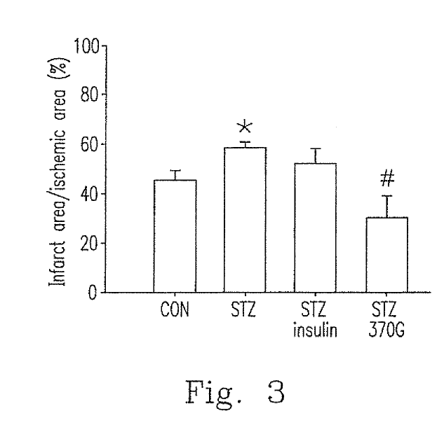

Please refer to FIG. 3, which shows the effects of insulin and the compound 370G on the infarct/ischemic areas of the cardiac muscle of the type I diabetic rats after the ligation/reperfusion treatment. The coronary artery of the rat heart is ischemic by the ligation, and the blood vessel is unclamped for the reperfusion, wherein some tissues of the local ischemic area will become necrotic. As shown in FIG. 3, the long-term treatment of the compound 370G could effectively improve the myocardial injury of the type I diabetic rats caused by the ligation/reperfusion (* denotes p<0.05 compared with the control group, and # denotes p<0.05 compared with the STZ group).

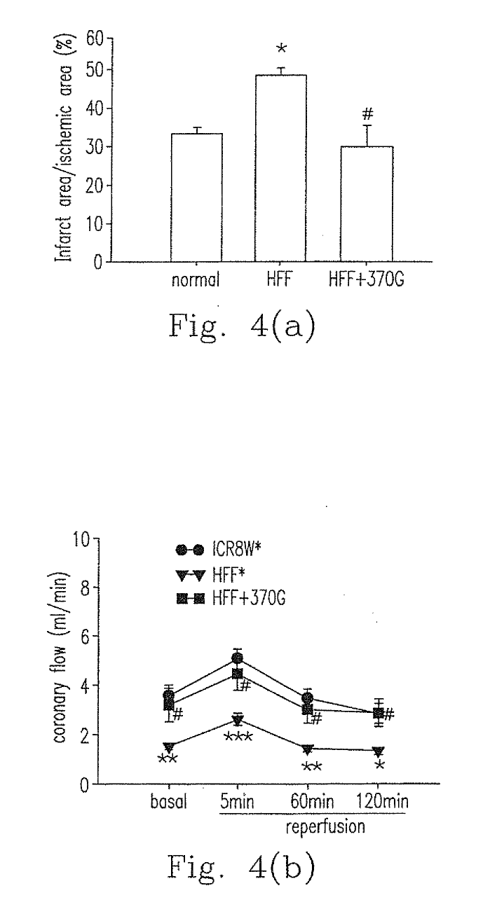

Please refer to FIG. 4(a), which shows the effects of the compound 370G on the infarct/ischemic areas of the cardiac muscle of the type II diabetic mice after the ligation/reperfusion treatment. The high-fat and high-fructose (HFF) induced type II diabetic mice are represented as the HFF group. The type II diabetic mice long-term treated with the compound 370G are represented as the HFF+370G group. As shown in FIG. 4(a), the long-term oral administration (e.g. two weeks) of the compound 370G (1-10 mg/kg once a day) could effectively improve the myocardial injury of the type II diabetic mice resulting from the myocardial ischemia-reperfusion (* denotes p<0.05 compared with the normal group, and # denotes p<0.05 compared with the HFF group).

Evaluations of the Flow Rate of the Coronary Artery

The method for detecting the flow rate of the coronary artery has been disclosed in Taiwan Patent application with the Publication No. 200812566.

Please refer to FIG. 4(b), which shows the effect of the compound 370G on the coronary flow rate of the type II diabetic mice after the ligation/reperfusion treatment. ICR8W group means the outbred mice representing the normal mice. As shown in FIG. 4(b), the long-term treatment of the compound 370G could effectively increase the coronary flow rate of the type II diabetic mice (* denotes p<0.05 compared with the normal group, and # denotes p<0.05 compared with the HFF group).

Evaluations of the Heart Failure in Laboratory Animal Model

1. The establishment of the abdominal aortic banding (AAB) ICR mice model

The eight-week-old male ICR mice are fed with water containing the penicillin, streptomycin and neomycin for preventing the surgical infection for three days before the surgery. The mice are anaesthetized by an intraperitoneal injection of a pentobarbital (75 mg/kg, i.p.), and then abdominal incisions are made to expose the abdominal aortas. The sterilized silk sutures are used to ligate aortas against blunted needles with a size 26G. The needles are then removed immediately to create a defined constriction. The abdominal cavity, muscles and skin are closed layer by layer with the silk sutures, and the wound is sterilized with the povidone iodine. The mice after the operation are placed in a warm recovery box and are fed with water containing antibiotics for three days.

This model is intended to imitate the clinical pathological phenomena of the heart failure caused by the hypertension. For the AAB ICR mice, since the artery pressure is increased, the heart workload is increased accordingly. After about eight weeks, an apparent hypertrophy of the heart would be caused, which can lead into heart failure over time. Therefore, this model is suitable to be a model for developing a drug for treating the heart failure.

2. The Experimental Process

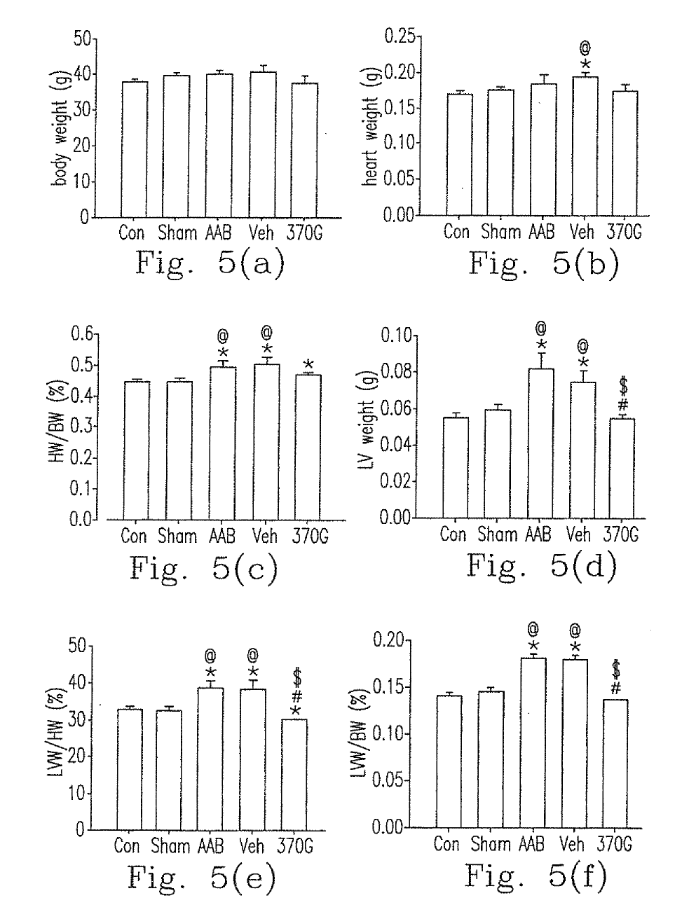

Mice are randomly divided into control group, sham-operated control group, AAB-operated group, AAB-operated group with the oral administration of vehicle, and AAB-operated group with the oral administration of the compound 370G, which are referred to as Con, Sham, AAB, Veh and 370G in FIGS. 5-9, respectively. Specifically, mice are subjected to either pressure overload generated by abdominal aorta banding operation (AAB group) or sham surgery (sham group), and one day after the operation, some of them are oral administrated once a day with 1 mg/kg 370G (370G group) or vehicle (0.5% tartaric acid and 4.6% glucose) (Veh group) for 2 months. After the administration for 2 months, the mice are killed for performing the hemodynamics analysis and the heart morphology analysis.

3. Detection of the Left Ventricular Hypertrophy

The mice are anaesthetized by an intraperitoneal injection of a pentobarbital (75 mg/kg), and then the body weight (BW) is measured. After the evaluation of the cardiac function by the carotid artery cannula, the mice are killed by cervical dislocation, and the heart is taken out of the body immediately. At the same time, the blood is sampled and centrifugated, and the supernatant liquid (serum) is stored at −80□. The taken-out heart is washed with normal saline, and the non-heart tissue is removed so that a heart weight (HW) is obtained. Then, the left ventricle is cut to get a left ventricle weight (LVW). The myocardial hypertrophy indices are expressed as heart weight/body weight (HW/BW), left ventricle weight/body weight (LVW/BW), and left ventricle weight/heart weight (LVW/HW), which are calculated to evaluate the level of the heart hypertrophy.

Please refer to FIGS. 5(a)-(f), which are diagrams respectively showing the BW, HW, HW/BW, LVW, LVW/HW and LVW/BW in five groups. For all experiments in FIGS. 5(a)-(f), the results are presented as Mean±SEM of n≧6. Body weight (FIG. 5(a)), heart weight (FIG. 5(b)) and left ventricle weight (FIG. 5(d)) are weighted from each group at 8 weeks after operation. The aortic constriction-induced cardiac hypertrophy level is evaluated by the myocardial hypertrophy indices, HW/BW, LVW/BW, and LVW/HW. The heart, especially left ventricle, hypertrophied at 8 weeks after the AAB operation and the oral administration of the compound 370G (1-10 mg/kg once a day) ameliorates the left ventricle hypertrophy significantly (* denotes p<0.05 compared with the Con group, @ denotes p<0.05 compared with the Sham group, # denotes p<0.05 compared with the AAB group and $ denotes p<0.05 compared with the Veh group).

4. Evaluation of the Cardiac Function

The mice are anaesthetized by an intraperitoneal injection of a pentobarbital (75 mg/kg), and then a PV catheter (112B-C009) is inserted via the carotid artery into left ventricle and connected to a pressure transducer. The pressure transducer is connected to an amplifier and the amplified signal is acquired by the Power Lab system so as to display/record the real-time data on/in a computer. The evaluated cardiac function includes LVDP, +dp/dt and −dp/dt.

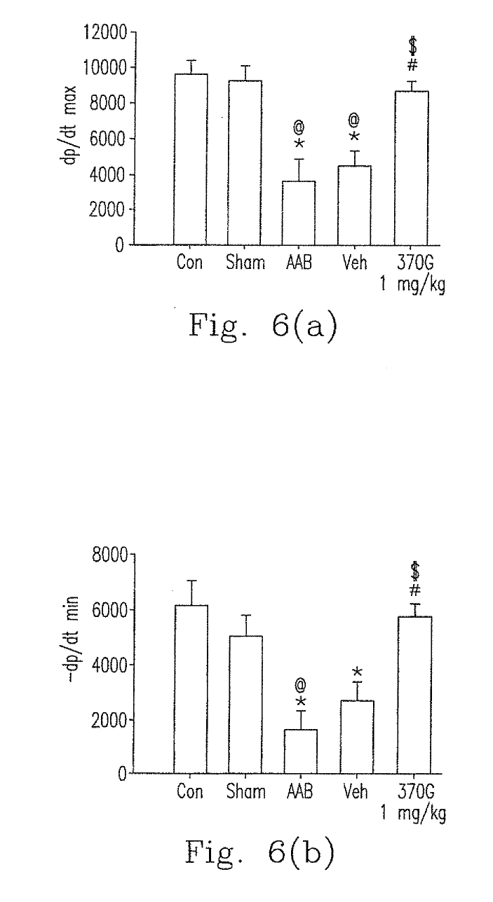

Please refer to FIGS. 6(a)-(b), which are diagrams showing the evaluated results of the cardiac function in five groups. For all experiments in FIGS. 6(a)-(b) the results are presented as means±SEM of n≧3. As shown, LV dysfunction is observed at 8 weeks after the AAB operation and the administration of the compound 370G (1 mg/kg once a day) improves the left ventricular function significantly (* denotes p<0.05 vs. Con, @ denotes p<0.05 vs. Sham; # denotes p<0.05 vs. AAB and $ denotes p<0.05 vs. Veh). Based on the above, it could be known that the oral administration with the compound 370G 1 mg/kg/day can ameliorate the cardiac dysfunction in abdominal aortic banding-induced heart failure mice.

Assay of the Angiotensin II in the Serum

Before this experiment, all the serum sample and reagents are dissolve to room temperature (18˜25° C.). 100 μL anti-angiotensin II antibody is added into each well of a plate coated with a secondary antibody, and the plate is incubated for 1.5 hours. The solution in each well is discarded, and each well is washed 4 times with 200 μl of 1× Wash Solution. 100 μl of Angiotensin II standard, positive control and serum sample are added into different wells. Wells are covered and incubated for 2.5 hours at room temperature or over night at 4° C. The solution in each well is discarded, and each well is washed 4 times with 200 μl of 1× Wash Solution. 100 μl of prepared HRP-Streptavidin solution is added into each well, and the plate is incubated for 45 minutes at room temperature. The solution in each well is discarded, and each well is washed 5 times with 200 μl of 1× Wash Solution. 100 μl of TMB One-Step Substrate Reagent is added into each well, and the plate is incubated for 30 minutes at room temperature in the dark. Finally, 50 μl of Stop Solution is added into each well, and the plate is read by a 96-well plate reader (VICTOR3 multilabel reader, Perkin-Elmer Life Sciences, Boston, Mass., USA) at 450 nm immediately. A standard curve of known concentration of Angiotensin II peptide is established and the concentration of Angiotensin II peptide (μg/ml) in the samples is calculated accordingly.

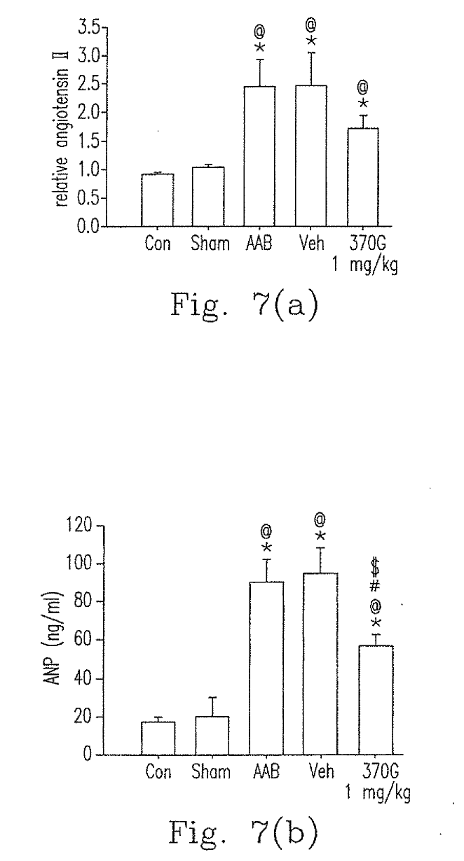

Please refer to FIG. 7(a), which is a diagram showing the effect of the compound according to the present invention on the changes in plasma levels of angiotensin II after the aortic constriction. For this experiment the results are presented as means±SEM of n≧6. As shown in FIG. 7(a), plasma angiotensin. II increases at 8 weeks after the AAB operation, and the administration of the compound 370G decreases the plasma levels of angiotensin II significantly (* denotes p<0.05 vs. Con, @ denotes p<0.05 vs. Sham; # denotes p<0.05 vs. AAB and $ denotes p<0.05 vs. Veh).

Assay of Atrial Natriuretic Peptide (ANP) in the Serum

Before this experiment, all the serum sample and reagents are dissolve to room temperature (18˜25° C.). 100 μL anti-ANP antibody is added into each well of a plate coated with a secondary antibody, and the plate is incubated for 1.5 hours. The solution in each well is discarded, and each well is washed 5 times with 200 μl of 1× Wash Solution. 100 μl of ANP standard, positive control and serum sample are added into different wells. Wells are covered and incubated for 2.5 hours at room temperature or over night at 4° C. The solution in each well is discarded, and each well is washed 4 times with 200 μl of 1× Wash Solution. 100 μl of a prepared HRP-Streptavidin solution is added into each well, and the plate is incubated for 45 minutes at room temperature. The solution in each well is discarded, and each well is washed 5 times with 200 μl of 1× Wash Solution. 100 μl of TMB One-Step Substrate Reagent is added into each well, and the plate is incubated for 30 minutes at room temperature in the dark. Finally, 50 μl of Stop Solution is added into each well, and the plate is read by a 96-well plate reader (VICTOR3 multilabel reader, Perkin-Elmer Life Sciences, Boston, Mass., USA) at 450 nm immediately. A standard curve of known concentration of ANP is established and the concentration of ANP (μg/ml) in the samples is calculated accordingly.

Please refer to FIG. 7(b), which is a diagram showing the effect of the compound according to the present invention on the changes in plasma levels of ANP after the aortic constriction. For this experiment the results are presented as means±SEM of n≧6. As shown in FIG. 7(b), plasma ANP increases at 8 weeks after the AAB operation, and the administration of the compound 370G decreases the plasma levels of ANP significantly (* denotes p<0.05 vs. Con, @ denotes p<0.05 vs. Sham; # denotes p<0.05 vs. AAB and $ denotes p<0.05 vs. Veh).

Paraffin Embedding, Sectioning and HE Stain

1. Fixation

Mice organs are removed from the body and put into a automatic fix machine, which could carry out the sample fixation within a predefined time period by automatically change the immersion solutions. The step order of the sample fixation is as follows: 1. fixation: 10% formalin for 1 hour and H₂O for 30 min; 2. dehydration: 80% EtOH for 1 hour, 95% EtOH for 1 hour, 95% EtOH for 2 hours, 100% EtOH for 1 hour, 100% EtOH for 1 hour and 100% EtOH for 2 hours; 3. clearing: xylene for 1 hour and xylene for 2 hours; and 4, infiltration: paraffin for 1 hour and paraffin for 2 hours.

2. Paraffin Embedding

The fixed sample is placed in a box, and the warm paraffin is added slowly into the box until the sample is embedded. The box is covered by its cover, and placed on a cold plate to solidify the paraffin. Finally, the box is stored in a refrigerator until the sectioning process is to be initiated.

3. Paraffin Sectioning

A rotary microtome is used to slice up the sample into sections with a thick less than 10 μm (preferably less than 5 μm). The sections are placed in the cold water to extend the paraffin, and the sections are attached to a glass slide. The glass slide is placed in a warm water bath and dried by placing into an oven.

4. HE Stain

The dried glass slide is stained by a histology staining method comprising the steps of: deparaffinizing sections: xylene for 5 min, xylene for 5 minutes and xylene for 5 minutes; rehydrating sections: 100% EtOH for 1 minute, 100% EtOH for 1 minute, 95% EtOH for 1 minute and 80% EtOH for 1 minute; staining the nuclei with hematoxylin for 2-15 minutes; washing in running water for 3-5 minutes; bluing in 0.25% aqueous ammonia for 10 seconds; washing the slides in running water for 5 minutes; stain the cytoplasm with Eosin for 25 seconds to 1 minute; washing for 5 seconds; dehydrating slides: 70% EtOH for 20 seconds, 80% EtOH for 20 seconds, 95% EtOH for 20 seconds and 100% EtOH for 20 seconds (repeated three times); clearing slides in xylene for 2 minutes 3 times.

Masson Trichrome Stain

The experiment comprises steps of deparaffinizing slides by deionized water, mordanting in preheated Bouin's solution at 56° C. for 15 minutes, cooling slides in tap water (18-26° C.), washing in running tap water to remove yellow color from sections, staining in the prepared Weigert's iron hematoxylin solution for 5 minutes, washing in running tap water for 5 minutes, rinsing in deionized water, staining in biebrich scarlet-acid fucshin for 5 minutes, rinsing in deionized water, placing slides in the prepared phosphotungstic/phosphomolybdic acid solution for 5 minutes, placing slides in aniline blue solution for 5 minutes, placing slides in 1% acetic acid for 2 minutes, discarding solutions, rinsing slides, dehydrating slides through alcohol, clearing in xylene and mounting slides.

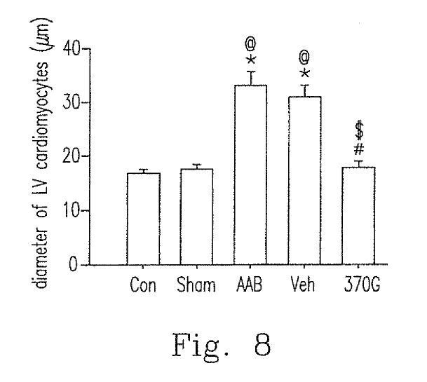

Please refer to FIG. 8, which is a diagram showing the effect of the compound according to the present invention on the changes in the cardiomyocyte diameter after the aortic constriction. For this experiment the results are presented as means±SEM of n≧3. The diameter of the cardiomyocyte is determined by the software, ImageQuant. As shown in FIG. 8, the diameters of cardiomyocytes from left ventricular increased at 8 weeks after the operation. The treatment of 370G once a day significantly decreases the diameters of cardiomyocytes from left ventricular (* denotes p<0.05 vs. Con, @ denotes p<0.05 vs. Sham; # denotes p<0.05 vs. AAB and $ denotes p<0.05 vs. Veh). Masson trichrome staining is used to estimate the level of fibrosis in left ventricular free wall. The abdominal aorta banding operation leads to severe fibrosis and the treatment of the compound 370G (1 mg/kg once a day) decreases the fibrosis (data not shown).

Lactic Dea Hydrogenase (LDH) Release Assay

The assay comprises the steps of adding 50 μl of a 1:10 dilution of serum sample to each well of a 96-well culture plate, adding 50 μl of Substrate Mix (CytoTox 96® Non-Radioactive Cytotoxicity Assay, Promega) into each well of the plate, covering the plate and incubating at room temperature for 30 minutes in the dark, adding 50 μl of Stop Solution to each well, and recording absorbance at 492 nm by a 96-well plate reader (VICTOR3 multilabel reader, Perkin-Elmer Life Sciences, Boston, Mass., USA).

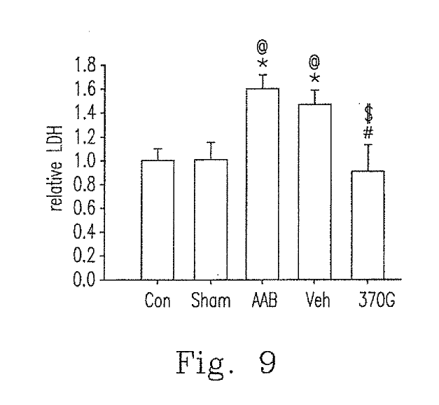

Please refer to FIG. 9, which is a diagram showing the effect of the compound according to the present invention on the changes in the plasma LDH level after the aortic constriction. For this experiment the results are presented as means±SEM of n≧6. As shown in FIG. 9, the plasma LDH increases at 8 weeks after the operation, and the treatment of the compound 370G decreases the release of LDH significantly (* denotes p<0.05 vs. Con, @ denotes p<0.05 vs. Sham; # denotes p<0.05 vs. AAB and $ denotes p<0.05 vs. Veh).

In view of the above experimental results, it could be known that the catechol-based derivatives of the present invention are indeed capable of preventing or treating the chronic heart diseases, especially the chronic heart failure. In a preferred embodiment, compounds of the invention are administered therapeutically, and preferably, prophylactically, to patients suffering from or in danger of suffering from cardiac hypertrophy disease, preferably pressure overload cardiac hypertrophy. In a preferred embodiment, a compound of the invention is administered with one or more anti-cardiac-hypertrophy drug. It is within the skill of those in the art to monitor and adjust the treatment or prophylactic regimen for treating or preventing chronic heart diseases.

The compounds disclosed in the present invention are safe and effective in treating or preventing chronic heart diseases or abnormal metabolism by the long-term oral administration at a dose of 1-10 mg/kg/day. Further, a single-dose toxicity study is conducted in mice by oral administration of a dose 100 mg/kg, but for the mice no significant toxicity is observed, and no example of abnormality nor death are recognized during the observation period of a week,

### Embodiments

1. A pharmaceutical composition for preventing or treating a chronic heart disease, comprising a compound of a formula (I):

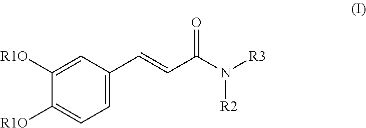

or a salt thereof, wherein:
R1 is one independently selected from a group consisting of a hydrogen, a methyl and an ethyl;
R2 is one of a hydrogen and a methyl; and
R3 is one selected from a group consisting of a hydrogen, (CH₂)ₙAr and (CH₂)ₙArR′R″, wherein n is one of 1 and 2, R′ and R″ is located at C-3 and C-4 positions, respectively, R′ is a hydrogen and R″ is one of a hydroxy, a fluorine, a bromine and a OMe, or R′+R″=—OCH₂O—; or
R2+R3 is one of

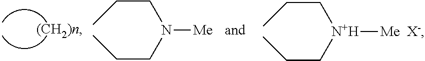

wherein n is one of 4 and 5.

2. The pharmaceutical composition of embodiment 1, wherein the chronic heart disease is caused by a hypertension.

3. The pharmaceutical composition of any of the preceding embodiments, wherein the chronic heart disease is caused by an abnormal metabolism.

4. The pharmaceutical composition of any of the preceding embodiments, wherein the chronic heart disease is a chronic heart failure.

5 The pharmaceutical composition of any of the preceding embodiments, further comprising at least one of a pharmaceutically acceptable carrier, a diluent and an excipient.

6. A method of treating a chronic heart disease, comprising:
administering a therapeutically effective amount of a pharmaceutical composition as claimed in embodiment 1 to a subject in need thereof.

7. The method of embodiment 6, further comprising:
alleviating a symptom of a left ventricular hypertrophy.

8. The method of any of the preceding embodiments, wherein the chronic heart disease is a chronic heart failure.

9. The method of any of the preceding embodiments, further comprising:
increasing a flow rate of a coronary artery.

10. The method of any of the preceding embodiments, further comprising:
reducing a necrosis area of a myocardium.

11. The method of any of the preceding embodiments, wherein the necrosis area of the myocardium is caused by an ischemia-reperfusion.

12. The method of any of the preceding embodiments, further comprising:
reducing an angiotensin concentration in a blood plasma.

13. The method of any of the preceding embodiments, further comprising:
reducing an atrial natriuretic peptide concentration in a blood plasma.

14. A use of a compound as described in embodiment 1 in the manufacture of a medicament for the prevention or treatment of a disease or condition ameliorated by reducing one of a necrosis area of a myocardium, an angiotensin concentration in a blood plasma and an atrial natriuretic peptide concentration in the blood plasma.

15. The use of embodiment 14, wherein the necrosis area of the myocardium is caused by an ischemia-reperfusion.

16. A compound of a formula (II):

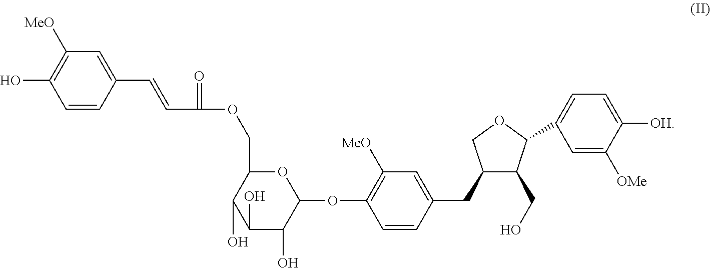

17. The compound of embodiment 16, being used for preventing or treating a chronic heart disease.

18. A pharmaceutical composition for preventing or treating a chronic heart disease, comprising a compound as described in embodiment 16.

19. The pharmaceutical composition of embodiment 18, further comprising at least one a pharmaceutically acceptable carrier a diluent and an excipient.

While the invention has been described in terms of what is presently considered to be the most practical and preferred embodiments, it is to be understood that the invention needs not be limited to the disclose embodiments. Therefore, it is intended to cover various modifications and similar arrangements included within the spirit and scope of the appended claims, which are to be accorded with the broadest interpretation so as to encompass all such modifications and similar structures.

What is claimed is:

1. A pharmaceutical composition for preventing or treating a chronic heart disease, comprising a compound of a formula (I): 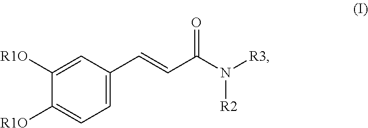
wherein:
R1 is one independently selected from a group consisting of a hydrogen, a methyl and an ethyl;
R2 is one of a hydrogen and a methyl; and
R3 is one selected from a group consisting of a hydrogen, (CH₂)ₙAr and (CH₂)ₙArR′R″, wherein n is one of 1 and 2, R′ and R″ is located at C-3 and C-4 positions, respectively, R′ is a hydrogen and R″ is one of a hydroxy, a fluorine, a bromine and a OMe, or R′+R″=—OCH₂O—; or
R2+R3 is one of 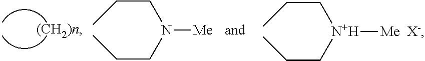
wherein n is one of 4 and 5.

2. A pharmaceutical composition as claimed in claim 1, wherein the chronic heart disease is caused by a hypertension.

3. A pharmaceutical composition as claimed in claim 1, wherein the chronic heart disease is caused by an abnormal metabolism.

4. A pharmaceutical composition as claimed in claim 1, wherein the chronic heart disease is a chronic heart failure.

5. A pharmaceutical composition as claimed in claim 1, further comprising one selected from a group consisting of a pharmaceutically acceptable carrier, a diluent, an excipient and a combination thereof.

6. A method of preventing or treating a chronic heart disease, comprising:
administering a therapeutically effective amount of a pharmaceutical composition as claimed in claim 1 to a subject in need thereof.

7. A method as claimed in claim 6, further comprising:
alleviating a symptom of a left ventricular hypertrophy by administering the pharmaceutical composition to the subject.

8. A method as claimed in claim 6, further comprising:
alleviating a symptom of a left ventricular hypertrophy.

9. A method as claimed in claim 6, wherein the chronic heart disease is a chronic heart failure by administering the pharmaceutical composition to the subject.

10. A method as claimed in claim 6, further comprising:
reducing a necrosis area of a myocardium by administering the pharmaceutical composition to the subject.

11. A method as claimed in claim 10, wherein the necrosis area of the myocardium is caused by an ischemia-reperfusion.

12. A method as claimed in claim 6, further comprising:
reducing a concentration of an angiotensin in a blood plasma.

13. A method as claimed in claim 6, further comprising:
reducing a concentration of an atrial natriuretic peptide in a blood plasma.

14. A compound of a formula (II):

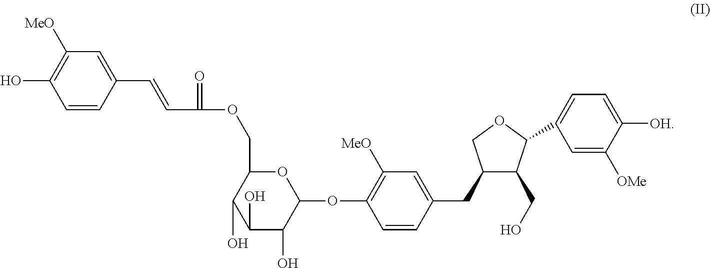

15. A compound as claimed in claim 14, being used for preventing or treating a chronic heart disease.

16. A compound as claimed in claim 15, wherein the chronic heart disease is caused by a hypertension.

17. A compound as claimed in claim 15, wherein the chronic heart disease is caused by an abnormal metabolism

18. A compound as claimed in claim 15, wherein the chronic heart disease is a chronic heart failure.

19. A pharmaceutical composition for preventing or treating a chronic heart disease, comprising a compound as claimed in claim 14.

20. A pharmaceutical composition as claimed in claim 19, further comprising one selected from a group consisting of a pharmaceutically acceptable carrier, a diluent, an excipient and a combination thereof
